(19) United States
(12) Patent Application Publication
(10) Pub. No.: US 2006/0127918 A1
(43) Pub. Date: Jun. 15, 2006

(54) Nucleic acid arrays

(75) Inventors: Mansoor S. Mohammed, Mission Viejo, CA (US); Natasa Dzidic, Laguna Beach, CA (US)

Correspondence Address: FOLEY & LARDNER LLP, P.O. BOX 80278, SAN DIEGO, CA, 92138-0278, US

(73) Assignee: Quest Diagnostics Investments Incorporated

(21) Appl. No.: 11/013,294

(22) Filed: Dec. 14, 2004

(51) Int. Cl. C12Q 1/68; C12M 1/34; C07H 21/04

(52) U.S. Cl. 435/6; 435/287.2; 536/25.32

(57) ABSTRACT

Described is a nucleic acid-containing substrate that is useful for nucleic acid hybridization methods, such as methods that utilize nucleic acid microarrays. The substrate contains nucleic acid that had not been covalently modified prior to having been contacted with the substrate and/or nucleic acid that is bound substantially non-covalently to the substrate. The nucleic acid remains associated with and/or bound to the substrate even after high stringency washings. Also described is a method for preparing the nucleic acid-containing substrate and methods for using the nucleic acid-containing substrate to detect or quantitate a target nucleic acid.

### FIELD OF THE INVENTION

The present invention relates to the manufacture and use of nucleic acid arrays also known as microarrays or biochips. In a particular aspect, the invention relates to such arrays prepared without covalently modified nucleic acid. The invention also relates to arrays that include a nucleic acid-containing substrate in which the nucleic acid may not be covalently bound.

### BACKGROUND OF THE INVENTION

Nucleic acid arrays are important in the biotechnology industry and related industries. Several useful applications for nucleic acid arrays procedures have been developed, including nucleic acid sequencing, gene expression analysis, and gene mutation analysis. One important application for nucleic acid arrays technology is the analysis of differential gene expression in which the expression of genes in different samples are compared and specific genes that are differentially expressed are identified. Typically, differential gene expression is analyzed by comparing a sample of interest to a control sample.

Nucleic acid arrays are also useful for array-based comparative genomic hybridization (“array-CGH”). Comparative genomic hybridization (“CGH”) is advantageous over conventional chromosome spread-based CGH techniques in that it provides improved quantitative accuracy and higher resolution as well as facilitating the analysis of samples. Array-CGH may be conducted with a variety of DNA probes including oligonucleotides (Lucito et al., Genome Research, 10:1726-1729, 2000); cDNA clones (Pollack et al., Nat. Genet. 23:41-46, 1999); bacterial artificial chromosomes (BACs) and E. coli P1 artificial chromosomes (PACs) (Solinas-Toldo et al., Genes Chromosomes Canc. 20:399-407, 1997). For the analysis of total genomic DNA, typically BACs and/or PACs provide the best performance (Albertson et al., Human Mol. Genet. 12:R145-152, 2003).

The process of manufacturing nucleic acid arrays involves depositing a plurality of nucleic acids (e.g., nucleic acid segments) in the form of “spots” at discrete locations of a solid surface. This process is commonly called “printing” a nucleic acid array. A variety of microarray equipment has been developed for printing arrays (e.g., BioRobotics Microgrid (Ann Arbor, Mich.) and others, collectively referred to as “arrayers”). The nucleic acids for printing may include oligonucleotides, reverse transcribed mRNA libraries (i.e., cDNA libraries), or large insert genomic clones (e.g., BACs).

Printing is normally conducted with chemically modified DNA so as to facilitate covalent attachment of the DNA to the solid surface. Covalent modification is believed to be necessary in order to retain the probes on the array during washing steps that normally following hybridization steps. Covalent modifications may include the addition of amino groups and/or thiol groups that can react with functional groups at the surface of a microarray substrate (e.g., activated ester groups) (Ramakrishnan et al., Nucl. Acids Res., Vol. 30, No. 7, (2002) e30). For example, 5′-NH₂(CH₂)₆-modified oligonucleotides (typically <˜100 nucleotides in length) may be synthesized for covalent attachment at the surface of a microarray substrate. However, modification of larger DNA molecules (e.g., BACs) may require more extensive protocols (e.g., treatment with a restriction enzyme and end-labeling with a nucleotide transferase).

### SUMMARY OF THE INVENTION

Disclosed is a nucleic-containing substrate that includes: (a) an organosilane-pretreated surface; (b) a polymer film cross-linked to the organosilane-pretreated surface; and (c) a nucleic acid molecule bound to one or more of the polymer film and the organosilane-pretreated surface. In preferred embodiments, the polymer film is formed from a polymer comprising reactive groups, and the nucleic acid molecule has not been covalently modified to facilitate covalent attachment at the reactive groups. The nucleic acid molecule may be associated with or bound to one or more of the polymer film and the organosilane-pretreated surface through covalent and/or noncovalent interactions.

In preferred embodiments, the nucleic acid molecule is at least about 250 nucleotides in length, and more preferably at least about 500 nucleotides in length. In one embodiment, the nucleic acid molecule is a DNA molecule present in the form of a bacterial artificial chromosome (BAC) or another suitable cloning vector (e.g., an E. coli P1 based artificial chromosome, a plasmid, a cosmid, and the like).

Typically, the bound nucleic acid molecule is present on the surface of the substrate at a concentration sufficient to detect a nucleic acid target molecule by nucleic acid hybridization methodology. For example, the nucleic acid molecule may be present at a concentration of at least about 500 copies/cm² on the surface of the substrate. More suitably the nucleic molecule is present on the surface of the substrate at a concentration of at least about 1000 copies/cm² and/or at least about 5000 copies/cm². The nucleic acid molecule preferably remains substantially attached to the substrate when subjected to washing under high stringency conditions (e.g., when the slide is washed with a low salt buffer optionally including a non-ionic detergent at a relatively high temperature). The term “substantially attached” as used herein means at least about 40% of the nucleic acid remains attached after high stringency washing, more preferably at least about 50% remains attached, more preferably at least 60% remains attached, more preferably at least about 70% remains attached, more preferably at least about 80% remains attached, more preferably at least about 90% remains attached, and more preferably at least about 95% remains attached.

The organosilane typically is a modified silane molecule that includes alkyl groups. In one embodiment, the organosilane includes alkyl groups with six or more carbon atoms and preferably ten or more carbon atoms. The organosilane may include alkoxy groups. The organosilane may also include halide groups.

The polymer preferably comprises reactive groups. Suitable reactive groups include electrophilic groups that react with nucleophilic groups under suitable conditions. For example, reactive groups may include amino-reactive groups (i.e., groups that react with the nitrogen atom of an amino group), thiol-reactive groups (i.e., groups that react with the sulfur atom of a thiol-group), hydroxyl-reactive groups (i.e., groups that react with the oxygen atom of a hydroxyl-group), and combinations thereof. In some embodiments, the polymer may include activated esters, epoxides, azlactones, activated hydroxyls, aldehydes, isocyanates, thioisocyanates, carboxylic acid chlorides, alkyl halides, maleimide, α-iodoacetamides, or combinations thereof. In one embodiment, the reactive group is an activated ester, and in particular, the activated ester may include an N-hydroxylsuccinimide ester.

The polymer may be synthesized from any suitable monomer, which may provide a polymeric backbone. The polymer may include one or more of acrylics, vinyls, nylons, polyurethanes and polyethers. In one embodiment, the polymer is a polyacrylic polymer.

The polymer preferably includes photoreactive groups that are capable of being cross-linked to the organosilane-pretreated surface of the substrate. Suitable photoreactive groups include photoreactive aryl ketones. In some embodiments, the photoreactive aryl ketones may include acetophenones, benzophenones, anthraquinones, anthrones, heterocyclic analogs of anthrones, and combinations thereof.

The nucleic acid-containing substrate may be configured as a nucleic acid microarray. The nucleic acid microarray may be suitable for performing comparative genomic hybridization analysis. In one embodiment the nucleic acid microarray comprises genomic DNA cloned in bacterial artificial chromosomes (BACs).

Also disclosed is a method for preparing a nucleic acid-containing substrate as described above. The method typically includes: (a) pretreating a surface of the substrate with a composition that includes an organosilane; (b) coupling a polymer to the organo-silane pretreated surface to form a polymer film; and (c) binding a nucleic acid molecule to one or both of the organosilane-pretreated surface and the polymer film. In preferred embodiments, the polymer film is formed from a polymer comprising reactive groups, and the nucleic acid has not been covalently modified to facilitate covalent attachment at the reactive groups. The nucleic acid molecule may be associated with and/or bound to one or more of the polymer film and the organosilane-pretreated surface through covalent and/or noncovalent interactions.

In preferred embodiments, the nucleic acid molecule is at least about 250 nucleotides in length. More preferably, the nucleic acid molecule is at least about 500 nucleotides in length, at least about 1000 nucleotides in length, or at least about 5000 nucleotides in length.

Also disclosed is a method for detecting the presence and/or amount of a target nucleic acid molecule in a sample that includes: a) contacting the target molecule with a nucleic acid-containing substrate, which is prepared as described above, under suitable conditions for hybridizing the target to the nucleic acid of the substrate; and b) detecting the presence of the target molecule bound to the substrate. In preferred embodiments, the nucleic acid-containing substrate is a nucleic acid microarray, and detection of the presence and/or amount of the nucleic acid target is performed using comparative genomic hybridization analysis.

### DETAILED DESCRIPTION OF THE PREFERRED EMBODIMENTS

Substrate

As used herein, a “substrate” is a solid support that provides a surface to which nucleic acid may be bound. For example, the substrate may be in the form of a slide or chip. A wide variety of organic and inorganic polymers, as well as other materials, both natural and synthetic, may be employed as the material for the substrate. For example, the entire substrate may comprise, or at least include a solid surface that comprises, nitrocellulose, nylon, glass, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition, gel-forming substance may be used, including proteins (e.g., gelatins), lipopolysaccharides, silicates, agarose and polyacrylamides. Where the substrate (or solid surface of the substrate) is porous, various pore sizes may be employed depending upon the nature of the system.

Alternative surfaces include derivatized surfaces such as chemically coated glass slides. One example, is the CodeLink™ Activated Slide sold by Amersham Biosciences (manufactured by SurModics, Inc. as 3D-Link™). These slides are coated with a novel 3-D surface chemistry comprising a long-chain, hydrophilic polymer containing amine-reactive groups, capable of reacting with and covalently immobilizing amine-modified DNA for microarrays. This polymer is formed from monomeric units that covalently crosslink to themselves and to the surface of the slide. The cross-linked polymer provides a film on the surface of the slide that is capable of immobilizing DNA such that the immobilized DNA is oriented away from the surface of the slide for improved hybridization. Suitable substrates and general considerations for preparing nucleic acid microarrays are described in U.S. Pat. No. 6,465,178 and in Sobek and Schlapbach, Substrate Architecture and Functionality: Defining the Properties and Performance of DNA, Peptide, Protein, and Carbohydrate Microarrays, MICROARRAY TECHNOLOGY, September 2004, pages 32-44.

As used herein, an “organosilane” is a compound having at least a central silicon atom covalently bonded to four substituents, where the substituents may be the same or different and at least one of which includes a carbon atom. Suitable substituents include straight chain or branched alkyl groups (e.g., alkyl groups that include at least 6 carbon atoms or more suitably at least 10 carbon atoms). In some embodiments, one or more substituents include a halide atom. For example, suitable organosilanes may include p-tolyldimethylchlorosilane (T-Silane) and N-decyldimethylchlorosilane. The organosilane may include an alkoxysilane.

The substrates described herein typically include a polymer film. The polymer typically includes a backbone that is synthetic or naturally occurring. Suitable polymer backbones include acrylics (e.g., those polymerized from hydroxyethyl acrylate, hydroxyethyl methacrylate, and the like), vinyls (e.g., those polymerized from polyvinylpyrrolidone, polyvinyl alcohols, and the like), nylons (e.g., those polymerized from polycaprolactam, polyhexamethylene adipamide, and the like), and polyethers (e.g., polyethylene oxides and the like). Suitable polymers are described in U.S. Pat. No. 6,465,178.

The polymers described herein typically include “reactive groups.” As used herein, “reactive group” means any chemical moiety capable of reacting with another chemical moiety under suitable conditions to form a covalent bond. “Reactive group” includes “thermochemically reactive groups” (i.e., groups having a reaction rate dependent on temperature). In one suitable embodiment, a “reactive group” includes an electrophilic group (e.g., an activated ester) capable of reacting with a nucleophilic group (e.g., an amino group) under suitable conditions to form a covalent bond. A “reactive group” also includes “photoreactive groups” (i.e., latent reactive groups that are responsive to various electromagnetic energy, and most suitably, those responsive to ultraviolet and visible electromagnetic energy). Suitable reactive groups including “thermochemically reactive groups” and “photoreactive groups” are described in U.S. Pat. No. 6,465,178.

Substrates (e.g., in the form of arrays), coatings (e.g., in the form of polymers), and/or reagents for preparing the same are described in U.S. Pat. Nos. 6,762,019; 6,709,712; 6,706,408; 6,669,994; 6,603,040; 6,562,136; 6,514,768; 6,514,734; 6,465,178; 6,406,754; 6,278,018; 6,254,634; 6,154,345; 6,121,027; 6,077,698; 6,007,833; and 5,858,653.

Nucleic Acid

The substrates described herein include bound nucleic acid. As used herein, “bound” or “immobilized” means that the nucleic acid is covalently and/or non-covalently coupled (either directly or indirectly) to the substrate, such that the nucleic acid is not substantially removed during a hybridization assay that includes one or more washing steps under high stringency conditions. High stringency conditions are known in the art and may include low salt concentrations (e.g., <4×SSC buffer and/or <2×SSC buffer), the presence of non-ionic detergent (e.g., 0.1% SDS), and/or relatively high temperatures (e.g., >55° C. and/or >70° C.).

As used herein, “nucleic acid” refers to segments or portions of DNA, cDNA, and/or RNA. Nucleic acid may also be derived or obtained from an originally isolated nucleic acid sample from any source (e.g., isolated from, purified from, amplified from, cloned from, reverse transcribed from sample DNA or RNA). “Genomic nucleic acid” refers to nucleic acid representing the genetic material of a plurality of chromosomes, preferably all chromosomes, contained in an organism. Genomic nucleic acid may be obtained from the nucleus of a cell, or recombinantly produced. Methods of purifying genomic DNA and/or RNA from a variety of samples are well-known in the art.

A nucleic acid segment may range in size from about 20 to about 200 nucleotides; about 200 to about 1,000 nucleotides; about 1,000 to about 100,000 nucleotides; or about 100,000 to about 1,000,000 nucleotides in length. Suitable nucleic acids for preparing the substrates described herein are typically at least about 250 nucleotides in length, and more typically at least about 500 nucleotides in length, at least about 1000 nucleotides in length, and/or at least about 5000 nucleotides in length. Nucleic acid of the present invention may be contained within a nucleic acid vector (e.g., plasmids, cosmids, etc.), or an artificial chromosome, such as a bacterial artificial chromosome (BAC) or an E. coli P1 derived artificial chromosome (PAC) as is known in the art.

The nucleic acid used to prepare the nucleic-containing substrates preferably is not covalently modified. As used herein, “covalent modification” includes methods of treating nucleic acid to provide a “non-naturally occurring reactive group.” A “non-naturally occurring reactive group” is a reactive group that is not present in the nucleic acid after standard purification and/or cloning techniques.

A “non-naturally occurring reactive group” may include an amino group provided at a non-natural position in the nucleic acid (e.g., as a 5′ substituent of the sugar moiety of a nucleotide of the nucleic acid). A “non-naturally occurring reactive group” may also include a thiol group. Typically, to facilitate covalent attachment to a substrate, nucleic acid is covalently modified at a free 5′ and/or 3′ end. For example, a nucleic acid may be covalent modified by providing a non-naturally occurring nucleotide at the 5′ end of the nucleic acid (e.g., a nucleotide including 5′-NH₂(CH₂)ₓ, where X is at least 6). However, “covalent modification” may include other methods for providing non-naturally occurring reactive groups. “Covalent modification” does not include denaturation of a nucleic acid sample (e.g., by heating and/or treating with a low salt buffer). A “covalent bond” or “covalent interaction” does not include ionic interactions, hydrophobic interactions, or interactions associated with van der Waals' forces. Typically, nucleic acids are covalently modified to facilitate covalent attachment to a substrate prior to detection of a target nucleic acid by hybridization (e.g., prior to CGH analysis).

Arrays and Nucleic Acid Printing

Nucleic acids can be immobilized to a substrate to prepare an array using methods disclosed herein, or using any other known methods for making nucleic acid arrays. Suitable methods that may be used in whole or in part or as variations thereof are disclosed, for example, in U.S. Pat. Nos. 6,562,565; 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston, Curr. Biol. 8:R171-R174, 1998; Schummer, Biotechniques 23:1087-1092, 1997; Kern, Biotechniques 23:120-124, 1997; Solinas-Toldo, Genes, Chromosomes & Cancer 20:399-407, 1997; Bowtell, Nature Genetics Supp. 21:25-32, 1999. See also published U.S. Patent Applications Nos. 20010018642; 20010019827; 20010016322; 20010014449; 20010014448; 20010012537; 20010008765.

The term “array,” “microarray,” “biochip,” or “chip” as used herein, refers to a plurality of “probe elements,” “target elements,” or “printed samples” or “spots”, each comprising a defined amount of one or more biological molecules, e.g., polypeptides, nucleic acid molecules, or probes, deposited at discrete locations on a substrate surface. As used herein, the term “nucleic acid array” refers to an array where the elements comprise nucleic acid samples. In preferred embodiments, the plurality of spots comprises nucleic acid samples, deposited at preferably at least about 50, at least about 100, at least about 300, or at least about 500 discrete locations on the surface. The plurality may comprise multiple repeats of the same nucleic acid segments, a variety of different nucleic acid segments, or combinations of the two to produce multiple spots (e.g., duplicate spots, triplicate spots, quadruplicate spots, quintuplicate spots, etc.). In one embodiment, the plurality comprises multiple repeats of the same nucleic acid segments to produce multiple spots.

The term “printing” as used herein, refers to the process of depositing nucleic acid samples onto discrete locations of a solid surface. The term “printing buffer” or “printing solution” as used herein, refers to a solution that is deposited to the array surface. Nucleic acid that is to be printed in an array is contacted with an appropriate printing solution prior to printing the array.

The term “salt” as used herein refers to one or more compounds that result from replacement of part or all of the acidic hydrogen of an acid by a metal, or an element acting like a metal.

As used herein, the term “arrayer” refers to equipment capable of printing an array by dispensing fluids at discrete locations on a solid surface. A variety of automated arrayers are available, for example the BioRobotics Microgrid, the Affymetrix Arrayer, the GeneMachines Omnigrid and the Packard Instrument Company Biochip Arrayer.

The term “spot” or “printed sample” as used herein, refers to the material that has been deposited at discrete locations of a solid surface by printing. For example, a printed sample or spot of a nucleic acid array refers to the individual locations where a nucleic acid containing solution has been deposited.

A suitable ionic printing solution may be aqueous or non-aqueous or a mixture of a aqueous liquid with a water miscible non-aqueous liquid. Ionic solutions are prepared by dissolving one or more ionic compounds into a liquid solution. Preferred ionic compounds include a salt or a buffer. In certain embodiments, the ionic solution comprises a suitable ionic compound at a concentration of at least 1 mM, at least 10 mM, at least 50 mM or at least 100 mM. In some embodiments, the ionic compound(s) in the printing solution is between 1-10 mM; 10-100 mM; 100-200 mM; or 200 mM-2M.

In one embodiment, the printing solution contains a Tris buffer or a salt thereof, the concentration being about 50 mM to about 300 mM, preferably about 75 mM to about 250 mM, more preferably about 100 to about 200 mM. In another embodiment, the printing solution contains EDTA or a salt thereof, the concentration being about 5 to about 30 mM, more preferably about 10 to about 20 mM. In a related embodiment, the ionic printing solution further comprises about 50 to about 100 mM NaOH. In another embodiment, the ionic printing solution comprises Tris, EDTA and sodium hydroxide. In a preferred embodiment, the ionic printing solution comprises 150 mM Tris, 15 mM EDTA, and 75 mM NaOH. In another embodiment, the printing solution comprises a salt of phosphate buffer, the concentration being about 50 mM to 300 mM or 100 mM to 200 mM and at a pH in the range of 6.0 to 7.0, 7.0 to 8.0, or 8.0 to 9.0. In yet another embodiment, the printing solution comprises 150 mM sodium phosphate buffer, pH 8.5.

Preferably, each printed nucleic acid sample on an array comprises a nucleic acid segment that is between about 1,000 (1 kB) and about 1,000,000 (1 MB) nucleotides in length, more preferably between about 100,000 (100 kB) and 300,000 (kB) nucleotides in length. In suitable embodiments, the printed nucleic acid sample comprises a nucleic acid segment that is at least about 250 nucleotides in length, and more suitably at least about 500 nucleotides in length, at least about 1000 nucleotides in length, and/or at least about 5000 nucleotides in length.

For CGH applications, an array may include a plurality of printed nucleic acid samples that together represents a chromosomal region of interest, a chromosome of interest, or an entire genome of interest. The plurality may reflect only portions of the total sequence. For example, an array of nucleic acid samples together representing a complete chromosome may include segments of 150 kb in length, each segment being the sole sample from every 3-4 MB of chromosomal sequence. In this case, the array can be stated to represent locations that are spaced at intervals about 3-4 megabases (MB) along the chromosome. In such case, arrays with higher resolution can be prepared where each sample of nucleic acid is taken from the target chromosome at intervals of about 2-3 megabases, or more preferably at intervals of about 1-2 megabases. As noted above, arrays may represent all chromosomes of a genome.

Hybridization

The methods used herein related to hybridization may incorporate all known methods and means (and variations thereof) of hybridization, including those useful for comparative genomic hybridization, see, e.g., U.S. Pat. Nos. 6,197,501; 6,159,685; 5,976,790; 5,965,362; 5,856,097; 5,830,645; 5,721,098; 5,665,549; 5,635,351. See also Diago, Am. J. Pathol. 158:1623-1631, 2001; Theillet, Bull. Cancer 88:261-268, 2001; Werner, Pharmacogenomics 2:25-36, 2001; Jain, Pharmacogenomics 1:289-307, 2000.

The term “hybridization” as used herein, refers to the pairing of substantially complementary nucleotide sequences (strands of nucleic acid) to form a duplex or heteroduplex through formation of hydrogen bonds between complementary base pairs in accordance with Watson-Crick base pairing. Hybridization is a specific, i.e., non-random, interaction between two complementary polynucleotides. Hybridization and the strength of hybridization (i.e., the strength of the association between the nucleic acids) is influenced by such factors as the degree of complementary between the nucleic acids, stringency of the conditions involved (e.g., temperature and salt concentration), and the Tₘ of the formed hybrid.

Generally, nucleic acid hybridizations comprise the following major steps: (1) immobilization of nucleic acids to a support to provide an immobilized probe; (2) pre-hybridization treatment to increase accessibility of the probe and to reduce nonspecific binding; (3) hybridization of a mixture of target nucleic acids to the probe; (4) post-hybridization washes to remove nucleic acid fragments not hybridized to the probe; and (5) detection of the target nucleic acid hybridized to the probe. The reagent used in each of these steps and their conditions for use may vary depending on the particular application. The terms “probe” and “target” may be used interchangeably. For example, the support may include an immobilized “target” nucleic acid to which a mixture of “probe” nucleic acids are hybridized.

The nucleic acid that is immobilized (e.g., as a probe) on a solid support or substrate as described herein remains substantially immobilized during standard hybridization steps, including high stringency wash conditions. For example, the nucleic acid remains substantially immobilized during washing conditions with 2% SSC buffer, 0.1% SDS, at temperatures of about 55° C. or greater (and more suitably at temperatures of about 70° C. or greater).

As used herein, the term “about” means “approximately” or “nearly.” In the context of numerical values, the term may be construed to estimate a value that is ±10% of the value or range recited.

### EXAMPLE

Preparation of BAC DNA Arrays Utilizing Non-covalently Modified DNA with the CodeLink™ Surface

Approximately 10 μg of BAC DNA was precipitated by adding approximately 1/12 volume 5M NaCl and 80% volume isopropanol and incubating the mixture at room temperature for 20 min. The mixture was centrifuged at 13200 rpm for 20 min to collect the precipitant (i.e., as a DNA pellet).

The supernatant was decanted and the DNA pellet was rinsed with 400 μl of 70% ethanol. The DNA pellet was resuspended in approximately 34 μl of 1× phosphate buffer and incubated at 65° C. for 1 hour. The resuspended DNA solution was vortexed and centrifuged to collect the DNA solution. The DNA in the solution was quantitated fluorometrically and adjusted to a concentration of about 100 ng/ul.

Approximately 32 μl of the DNA was aliquotted into a new Eppendorf tube. The DNA then was fragmented by subjecting the DNA solution to ultrasonic processing for 2 minutes at 100 amps. To confirm fragmentation of the DNA within a range of about 500-10,000 base pairs, a 2 μl sample of the fragmented DNA solution was subjected to electrophoresis on a 1% agarose gel.

The fragmented DNA was denatured by heating the DNA at 100° C. in a heat block for 10 minutes. The denatured DNA sample was rapidly cooled by placing the sample in a ice/ethanol slurry for 5 minutes.

The tubes were vortexed and centrifuged to collect the DNA sample, which was transferred to printing plates in a predetermined sample layout. The DNA sample was printed on the active surface of CodeLink™ slides (Amersham Biosciences) as outlined in the MicroGrid Operation Manual (Genomic Solutions).

The slides were scanned with an Axon laser scanner (Axon Instruments) at 650/990 Cy5/Cy3 PMT settings to verify the concentration and quality of the printed DNA using GenePix software. Individual BAC element intensity measurements of printed arrays were equivalent to those typically observed for covalently-modified and bound DNA (i.e., 500-5000 GenePix units).

The printed slides with non-covalently modified DNA were incubated overnight in a 75% humidity chamber and stored in a desiccator until use for hybridization analysis. The non-covalently modified DNA applied to the CodeLink™ slide withstood the highest stringency washings appropriate for CGH arrays. CGH analysis using CodeLink™ slides prepared as described above (using non-covalently modified DNA) or prepared as described by Amersham Biosciences (using covalently modified DNA) produced equivalent results.

All references, patents, and/or applications cited in the specification are indicative of the level of skill of those skilled in the art to which the invention pertains, and are incorporated by reference in their entireties, including any tables and figures, to the same extent as if each reference had been incorporated by reference in its entirety individually.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

What is claimed is:

1. A nucleic acid-containing substrate comprising:
a) an organosilane-pretreated surface;
b) a polymer film cross-linked to the organosilane-pretreated surface, wherein the polymer film is formed from a polymer comprising one or more reactive groups; and
c) a nucleic acid molecule bound to one or more of the polymer film and the organosilane-pretreated surface, wherein the nucleic acid molecule has not been covalently modified to facilitate covalent attachment to the reactive groups and wherein the nucleic acid molecule is at least about 250 nucleotides in length.

2. The nucleic acid-containing substrate of claim 1, wherein the nucleic acid molecule is at least about 500 nucleotides in length.

3. The nucleic acid-containing substrate of claim 1, wherein the nucleic acid molecule is present as a bacterial artificial chromosome.

4. The nucleic acid-containing substrate of claim 1, wherein the bound nucleic acid molecule is present at a concentration sufficient for detecting a nucleic acid target in a hybridization assay.

5. The nucleic acid-containing substrate of claim 4, wherein the bound nucleic acid molecule is present at a concentration of at least about 500 copies/cm².

6. The nucleic acid-containing substrate of claim 1, wherein the organosilane pretreated surface comprises alkyl groups.

7. The nucleic acid-containing substrate of claim 6, wherein the alkyl groups comprise ten or more carbon atoms.

8. The nucleic acid-containing substrate of claim 1, wherein the polymer comprises reactive groups selected from amino-reactive groups, thiol-reactive groups, hydroxyl reactive groups, and mixtures thereof.

9. The nucleic acid-containing substrate of claim 1, wherein the polymer comprises reactive groups selected from the group consisting of activated esters, epoxides, azlactones, activated hydroxyls, aldehydes, isocyanates, thioisocyanates, carboxylic acid chlorides, alkyl halides, maleimide, and α-iodoacetamide.

10. The nucleic acid-containing substrate of claim 9, wherein the selected reactive group is an activated ester.

11. The nucleic acid-containing substrate of claim 10, wherein the activated ester is an N-hydroxylsuccinimide ester.

12. The nucleic acid-containing substrate of claim 1, wherein the polymer comprises one or more of acrylics, vinyls, nylons, polyurethanes and polyethers.

13. The nucleic acid-containing substrate of claim 1, wherein the polymer comprises photoreactive groups capable of being cross-linked to the organosilane-pretreated surface.

14. The nucleic acid-containing substrate of claim 13, wherein the photoreactive groups are photoreactive aryl ketones.

15. The nucleic acid-containing substrate of claim 14, wherein the photoreactive aryl ketones are selected from the group consisting of acetophenone, benzophenone, anthraquinone, anthrone, heterocyclic analogs of anthrone, and mixtures thereof.

16. The nucleic acid-containing substrate of claim 1, wherein the substrate is in the form of a nucleic acid-containing microarray.

17. A nucleic acid-containing substrate suitable for comparative genomic hybridization, comprising:
a) an organosilane-pretreated surface;
b) a polymer film cross-linked to the organosilane-pretreated surface; and
c) a nucleic acid molecule bound substantially non-covalently to one or more of the polymer film and the organosilane-pretreated surface, wherein the bound nucleic acid molecule is present at a concentration sufficient for detecting a nucleic acid target in a hybridization assay.

18. The nucleic acid-containing substrate of claim 17, wherein the polymer film is formed from a polymer comprising one or more reactive groups.

19. The nucleic acid-containing substrate of claim 17, wherein the nucleic acid molecule is at least about 250 nucleotides in length.

20. The nucleic acid-containing substrate of claim 17, wherein the nucleic acid is present as a bacterial artificial chromosome.

21. The nucleic acid-containing substrate of claim 17, wherein the bound nucleic acid molecule is present at a concentration of at least about 500 copies/cm².

22. The nucleic acid-containing substrate of claim 17, wherein the organosilane pretreated surface comprises alkyl groups.

23. The nucleic acid-containing substrate of claim 22, wherein the alkyl groups comprise ten or more carbon atoms.

24. The nucleic acid-containing substrate of claim 18, wherein the polymer comprises reactive groups selected from amino-reactive groups, thiol-reactive groups, hydroxyl reactive groups, and mixtures thereof.

25. The nucleic acid-containing substrate of claim 18, wherein the polymer comprises reactive groups selected from the group consisting of activated esters, epoxides, azlactones, activated hydroxyls, aldehydes, isocyanates, thioisocyanates, carboxylic acid chlorides, alkyl halides, maleimide, and a-iodoacetamide.

26. The nucleic acid-containing substrate of claim 25, wherein the selected reactive group is an activated ester.

27. The nucleic acid-containing substrate of claim 26, wherein the activated ester is an N-hydroxylsuccinimide ester.

28. The nucleic acid-containing substrate of claim 18, wherein the polymer comprises one or more of acrylics, vinyls, nylons, polyurethanes and polyethers.

29. The nucleic acid-containing substrate of claim 18, wherein the polymer comprises photoreactive groups capable of being cross-linked to the organosilane-pretreated surface.

30. The nucleic acid-containing substrate of claim 29, wherein the photoreactive groups are photoreactive aryl ketones.

31. The nucleic acid-containing substrate of claim 30, wherein the photoreactive aryl ketones are selected from the group consisting of acetophenone, benzophenone, anthraquinone, anthrone, heterocyclic analogs of anthrone, and mixtures thereof.

32. The nucleic acid acid-containing substrate of claim 17, wherein the substrate is in the form of nucleic acid containing microarray.

33. A method for preparing a nucleic acid-containing substrate comprising:
a) pretreating a surface of the substrate with a composition that includes an organosilane;
b) coupling a polymer to the organosilane pretreated surface to form a polymer film, wherein the polymer includes reactive groups; and
c) contacting a nucleic acid molecule to one or both of the organosilane-pretreated surface and the polymer film, wherein the nucleic acid molecule has not been covalently modified to facilitate covalent attachment to the reactive groups and the nucleic acid molecule is at least about 250 nucleotides in length.

34. The method of claim 33, wherein the nucleic acid molecule is at least about 500 nucleotides in length.

35. The method of claim 33, wherein the bound nucleic acid molecule is present at a concentration sufficient for detecting a nucleic acid target molecule in a hybridization assay.

36. The method of claim 33, wherein the bound nucleic acid molecule is present at a concentration of at least about 500 copies/cm².

37. The method of claim 33, wherein the bound nucleic acid molecule is present in a bacterial artificial chromosome.

38. The method of claim 33, wherein the organosilane includes alkyl groups comprising ten or more carbon atoms.

39. The method of claim 33, wherein the polymer comprises reactive groups selected from amino-reactive groups, thiol-reactive groups, hydroxyl reactive groups, and mixtures thereof.

40. The method of claim 33, wherein the polymer comprises reactive groups selected from the group consisting of activated esters, epoxides, azlactones, activated hydroxyls, aldehydes, isocyanates, thioisocyanates, carboxylic acid chlorides, alkyl halides, maleimide, and α-iodoacetamide.

41. The method of claim 40, wherein the selected reactive group is an activated ester.

42. The method of claim 41, wherein the activated ester is an N-hydroxylsuccinimide ester.

43. The method of claim 33, wherein the polymer film is formed from one or more of acrylics, vinyls, nylons, polyurethanes, and polyethers.

44. The method of claim 33, wherein the polymer film is formed from a polymer that comprises photoreactive groups capable of being cross-linked to the organosilane-pretreated surface.

45. The method of claim 44, wherein the photoreactive groups are photoreactive aryl ketones.

46. The method of claim 45, wherein the photoreactive aryl ketones are selected from the group consisting of acetophenone, benzophenone, anthraquinone, anthrone, heterocyclic analogs of anthrone, and mixtures thereof.

47. The method of claim 33, wherein coupling a polymer to the organosilane pretreated surface to form a polymer film comprises subjecting the polymer and the surface to ultraviolet electromagnetic energy.

48. The method of claim 33, further comprising subjecting the nucleic acid-containing substrate to ultraviolet electromagnetic energy subsequent to contacting a nucleic acid molecule to one or both of the organosilane-pretreated surface and the polymer film.

49. A method for detecting the presence or amount of a nucleic acid target molecule in a sample, the method comprising contacting the nucleic acid target molecule with a nucleic acid probe molecule present on a substrate under hybridization conditions, and determining if the nucleic acid target molecule has hybridized to the nucleic acid probe molecule, wherein the substrate is as described in claim 1.

50. The method of claim 49, wherein the nucleic acid probe molecule is at least about 500 nucleotides in length.

51. The method of claim 49, wherein the nucleic acid probe molecule is present at a concentration of at least about 500 copies/cm² on a surface of the substrate.

52. The method of claim 49, wherein the nucleic acid probe molecule is present as a bacterial artificial chromosome.

53. The method of claim 49, wherein the method involves competitive genomic hybridization.

54. A method for detecting the presence or amount of a nucleic acid target molecule in a sample, the method comprising contacting the nucleic acid target molecule with a nucleic acid probe molecule present on a substrate under hybridization conditions, and determining if the nucleic acid target molecule has hybridized to the nucleic acid probe molecule, wherein the substrate is as described in claim 17.

55. The method of claim 54, wherein the nucleic acid probe molecule is at least about 500 nucleotides in length.

56. The method of claim 54, wherein the nucleic acid probe molecule is present at a concentration of at least about 500 copies/cm² on a surface of the substrate.

57. The method of claim 54, wherein the nucleic acid probe molecule is present as a bacterial artificial chromosome.

58. The method of claim 54, wherein the method involves competitive genomic hybridization.
